(19) United States
(12) Patent Application Publication
(10) Pub. No.: US 2009/0191394 A1
(43) Pub. Date: Jul. 30, 2009

(54) Oarganic Compound, Optical Crystal Film and Method of Production Thereof

(76) Inventors: Pavel I. Lazarev, London (GB); Elena N. Sidorenko, Moscow (RU)

Correspondence Address: Fay Sharpe LLP, 1228 Euclid Avenue, 5th Floor, The Halle Building, Cleveland, OH, 44115, US

(21) Appl. No.: 12/083,260

(22) PCT Filed: Oct. 9, 2006
(86) PCT No.: PCT/GB2006/003754

§ 371 Date: Mar. 3, 2009

(30) Foreign Application Priority Data
Oct. 7, 2005 (GB) 0520489.6

(51) Int. Cl. B32B 17/06; C07D 241/38; B05D 3/00

(52) U.S. Cl. 428/220; 544/342; 428/426; 427/384; 427/299

(57) ABSTRACT

The present invention is related to the synthesis of acenaphthoquinoxaline sulfonamide derivatives and the manufacture of optical crystal films based on these compounds. Said acenaphthoquinoxaline sulfonamide heterocyclic derivatives have the general structural formula: where n is 1, 2 or 3; X is an acid group; m is 1, 2 or 3; Y is a counterion selected from the list consisting of H⁺, NH4⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH —OCH₃, —OC₂H₅, —OCOCH₃, —OCN, —SCN, —NH₂, —NHCOCH₃, —CONH₂; and z is 1, 2, 3 or 4.

The present invention relates generally to the field of organic chemistry and particularly to the organic crystal films with phase-retarding properties for displays. More specifically, the present invention is related to the synthesis of heterocyclic acenaphthoquinoxaline sulfonamide derivatives and the manufacture of optical crystal films based on these compounds.

In connection with polarization, compensation and retardation layers, films, or plates described in the present application, the following definitions of terms are used throughout the text.

The definition of the thin optical films is related to wavelength of light and defines thin films as films with thickness comparable to half of the wavelength of light in the region of the electromagnetic spectrum in which they are intended to operate.

The term optical axis refers to a direction in which propagating light does not exhibit birefringence.

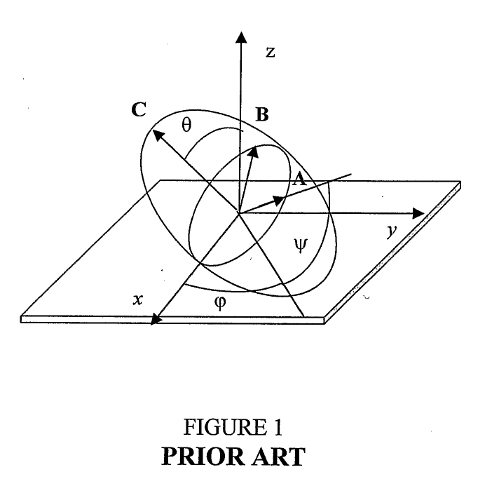

Any optically anisotropic medium is characterized by its second-rank dielectric permittivity tensor. The classification of compensator plates is tightly connected to orientations of the principal axes of a particular permittivity tensor with respect to the natural coordinate frame of the plate. The natural xyz coordinate frame of the plate is chosen so that the z axis is parallel to the normal direction and the xy plane coincides with the plate surface. FIG. 1 demonstrates the general case when the principal axes (A, B, C) of the permittivity tensor are arbitrarily oriented relative to the xyz frame.

Orientations of the principal axes can be characterized using three Euler's angles (θ, φ, ψ) which, together with the principal permittivity tensor components (ε_{A}, ε_{B}, ε_{C}), uniquely define various types of optical compensators (FIG. 1). The case when all the principal components of the permittivity tensor have different values corresponds to a biaxial compensator, whereby the plate has two optical axes. For instance, in the case of ε_{A}<ε_{B}<ε_{C}, these optical axes are in the plane of C and A axes on both sides from the C axis. In the uniaxial limit, when ε_{A}≈ε_{B} (ε_{A} is approximately equal to ε_{B}), we have a degenerate case when the two axes coincide and the C axis is a single optical axis.

The zenith angle θ between the C axis and the z axis is most important in the definitions of various types of optical compensators. There are several important types of compensator plates, which are most frequently used in practice.

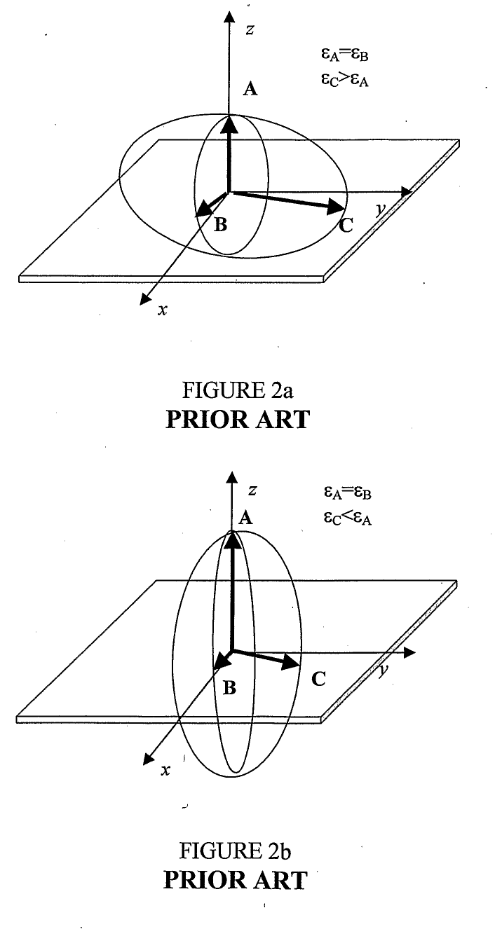

An uniaxial A-plate is defined by the Euler angle θ=π/2 and by the condition ε_{A}=ε_{B}≠ε_{C}. In this case, the principal C axis (extraordinary axis) occurs in the plane of the plate (xy plane), while the A axis (ordinary axis) is normal to the plate surface (due to the uniaxial degeneracy, the orthogonal orientations of A and B axes can be chosen arbitrarily in the plane that is normal to the xy surface). FIG. 2 shows the orientation of the principal axes of a particular permittivity tensor with respect to the natural coordinate frame of the positive (a) and negative (b) A-plate. The A-plates can be either positive (ε_{A}=ε_{B}<ε_{C}) or negative (ε_{A}=ε_{B}>ε_{C}).

In the general case, when the permittivity tensor components (ε_{A}, ε_{B}, and ε_{C}) are complex values, the principal permittivity tensor components (ε_{A}, ε_{B}, and ε_{C}), the refractive indices (na, nb, and nc), and the absorption coefficients (ka, kb, and kc) obey the following relations: na=Re[(ε_{A})^{1/2}], nb=Re[(ε_{B})^{1/2}], nc=Re[(ε_{C})^{1/2}], ka=lm[(ε_{A})^{1/2}], kb=lm[(ε_{B})^{1/2}], kc=lm[(ε_{C})^{1/2}].

Liquid crystals are widely used in electronic optical displays. In such display systems, a liquid crystal cell is typically situated between a pair of polarizer and analyzer plates. The incident light is polarized by the polarizer and transmitted through a liquid crystal cell, where it is affected by the molecular orientation of the liquid crystal that can be controlled by applying a bias voltage across the cell. Then, the altered fight is transmitted through the analyzer. By employing this scheme, the transmission of light from any external source, including ambient light, can be controlled. The energy required to provide for this control is generally much lower than that required for controlling the emission from luminescent materials used in other display types such as cathode ray tubes (CRTs). Accordingly, liquid crystal technology is used in a number of electronic imaging devices, including (but not limited to) digital watches, calculators, portable computers, and electronic games, for which small weight, low power consumption, and long working life are important.

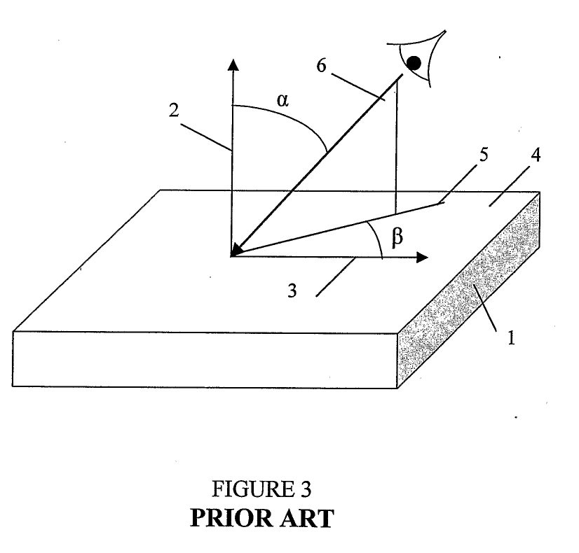

The contrast, color reproduction (color rendering), and stable gray scale intensity gradation are important quality characteristics of electronic displays, which employ liquid crystal technology. The primary factor determining the contrast of a liquid crystal display (LCD) is the propensity for light to “leak” through liquid crystal elements or cells, which are in the dark or “black” pixel state. In addition, the optical leakage and, hence, the contrast of an LCD also depend on the direction from which the display screen is viewed. Typically, the optimum contrast is observed only within a narrow viewing angle range centered about the normal (α=0) to the display and falls off rapidly as the polar viewing angle α is increased. Viewing direction herein is defined as a set of polar viewing angle α and azimuthal viewing angle (β) as shown in FIG. 3 with respect to a liquid crystal display 1. The polar viewing angle α is measured from display normal direction 2 and the azimuthal viewing angle (β) spans between an appropriate reference direction 3 in the plane of the display surface 4 and the projection 5 of viewing arrow 6 onto the display surface 4. Various display image properties such as contrast ratio, color reproduction, and image brightness are functions of the angles α and β. In color displays, the leakage problem not only decreases the contrast but also causes color or hue shifts with the resulting degradation of color reproduction.

LCDs are replacing CRTs as monitors for television (TV) sets, computers (such as, for example, notebook computers or desktop computers), central control units, and various devices, for example, gambling machines, electro-optical displays, (such as displays of watches, pocket calculators, electronic pocket games), portable data banks (such as personal digital assistants or of mobile telephones). It is also expected that the number of LCD television monitors with a larger screen size will sharply increase in the near future. However, unless problems related to the effect of viewing angle on the coloration, contrast degradation, and brightness inversion are solved, the replacement of traditional CRTs by LCDs will be limited.

The type of optical compensation required depends on the type of display used in each particular system. In a normally black display, the twisted nematic cell is placed between polarizers whose transmission axes are parallel to one another and to the orientation of the liquid crystal director at the rear surface of the cell (i.e., at the side of the cell away from the viewer). In the unenergized state (zero applied voltage), normally incident light from the backlight system is polarized by the first polarizer and transmitted through the cell with the polarization direction rotated by the twist angle of the cell. The twist angle is set to 90 DEG so that the output polarizer blocks this light. Patterns can be written in the display by selectively applying a voltage to the portions of the display which are to appear illuminated. However, when viewed at large angles, the dark (unenergized) areas of a normally black display will appear bright because of the angle-dependent retardation effect for the light rays passing through the liquid crystal layer at such angles, whereby off-normal incident light exhibits angle-dependent change of the polarization. The contrast can be restored by using a compensating element which has an optical symmetry similar to that of the twist cell but produces a reverse effect. One method consists in introducing an active liquid crystal layer containing a twist cell of reverse helicity. Another method is to use one or more compensators of the A-plate retarder type. These compensation methods work because the compensation element has the same optical symmetry as that of the twist nematic cell: both are made of uniaxial birefringent materials having the extraordinary axis orthogonal to the normal light propagation direction. These approaches to compensation have been widely utilized because of readily available materials with the required optical symmetry.

Thus, the technological progress poses the task of developing optical elements based on new materials with desired controllable properties. In particular, the necessary optical element in modern visual display systems is an optically anisotropic film that is optimised for the optical characteristics of an individual display module.

Various polymer materials are known in the prior art, which are intended for use in the production of optically anisotropic films. Films based on these polymers acquire optical anisotropy through uniaxial extension and coloring with organic dyes or iodine. Poly(vinyl alcohol) (PVA) is among commonly used polymers for this purpose. However, a low thermal stability of PVA based films limits their applications. PVA based films are described in greater detail in Liquid Crystals—Applications and Uses, B. Bahadur (ed.), World Scientific, Singapore—New York (1990), Vol. 1, p. 101.

Organic dichroic dyes are a recently developed class of materials currently gaining prominence in the manufacture of optically anisotropic films with desirable optical and working characteristics. Films based on these materials are formed by applying an aqueous liquid crystal (LC) solution of supramolecules formed by dye molecules onto a substrate surface with the subsequent evaporation of water. The applied films are rendered anisotropic either by preliminary mechanical orientation of the substrate surface or by applying external mechanical, electromagnetic, or other orienting forces to the LC film material on the substrate.

Liquid crystal properties of dye solutions are well known. In recent years, use of liquid crystals based on such dye solutions for commercial applications, such as LCDs and glazing coatings, has received much attention.

Dye supramolecules form lyotropic liquid crystals (LLCs). Substantial molecular ordering or organization of dye molecules in the form of columns allows such supramolecular LC mesophases to be used for obtaining oriented, strongly dichroic films.

Dye molecules forming supramolecular LC mesophases possess the following properties. These dye molecules contain functional groups located at their periphery, which impart water-soluble properties to these molecules. Organic dye mesophases are characterized by specific structures, phase diagrams, optical properties and solubility properties as described in greater detail in J. Lydon, Chromonics, in Handbook of Liquid Crystals, Wiley VCH, Weinheim (1998), Vol. 2B, p. 981-1007 (see also references therein).

Anisotropic films characterized by high optical anisotropy can be formed from LLC systems based on dichroic dyes. Such films exhibit the properties of E-type polarizers (due to light absorption by supramolecular complexes). Organic conjugated compounds with general molecular structure similar to dye molecules but without absorption in visible area of light spectrum can be used as retarders and compensators.

Retarders and compensators are films with phase-retarding properties in spectral regions where absorption is absent. Phase-retarding or compensating properties of such films are determined by their double refraction properties known as birefringence (Δn):

Δn=|nₒ−nₑ|,

which is the difference of refractive indices for the extraordinary wave (nₑ) and the ordinary wave (nₒ). The nₑ and nₒ values vary depending on the orientation of molecules in a medium and the direction of light propagation. For example, if the direction of propagation coincides with the optical or crystallographic axis, the ordinary polarization is predominantly observed. If the light propagates in the perpendicular direction or at some angle to the optical axis, the light emerging from the medium will separate into extraordinary and ordinary components.

It is also important that, in addition to the unique optical properties, the films based on organic aromatic compounds are characterized by high thermal stability and radiation stability (photostability).

Extensive investigations aimed at developing new methods of fabricating dye-based films through variation of the film deposition conditions have been described in U.S. Pat. Nos. 5,739,286 and 6,174,394 and in published patent application EP 961138. Of particular interest is the development of new compositions of lyotropic liquid crystals utilizing modifying, stabilizing, surfactant and/or other additives in the known compositions, which improve the characteristics of LC films.

There is increasing demand for anisotropic films with improved selectivity in various wavelength ranges. Films with different optical absorption maxima over a wide spectral interval ranging from infrared (IR) to ultraviolet (UV) regions are required for a variety of technological applications. Hence, much recent research attention has been directed to the materials used in the manufacturing of isotropic and/or anisotropic birefringent films, polarizers, retarders or compensators (herein collectively referred to as optical materials or films) for LCD and telecommunications applications, such as (but not limited to) those described in P. Yeh, Optical Waves in Layered Media, New York, John Wiley & Sons (1998) and P. Yeh, and C. Gu, Optics of Liquid Crystal Displays, New York, John Wiley & Sons, (1999).

It has been found that ultrathin birefringent films can be fabricated using the known methods and technologies to produce optically anisotropic films composed of organic dye LLC systems. In particular, the manufacture of thin crystalline optically anisotropic films based on disulfoacids of the red dye Vat Red 14 has been described by P. Lazarev and M. Paukshto, Thin Crystal Film Retarders (In: Proceeding of the 7th International Display Workshops, Materials and Components, Kobe, Japan, Nov. 29-Dec. 1 (2000), pp. 1159-1160) as cis- and trans-isomeric mixtures of naphthatenetetracarboxyilc acid dibenzimidazole:

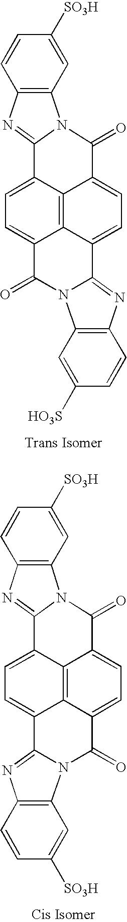

This technology makes it possible to control the direction of the crystallographic axis of a film during application and crystallization of LC molecules on a substrate (e.g., on a glass plate). The obtained films have uniform compositions and high molecular and/or crystal ordering with a dichroic ratio of approximately K_{d}˜28, which makes them useful optical materials, in particular, for polarizers, retarders, and birefringent films or compensators.

Thin birefringent films transparent in the visible spectral range have been obtained based on disodium chromoglycate (DSCG):

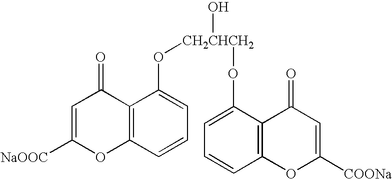

The anisotropy of oriented films made of DSCG is not very high: a difference in the refractive indices Δn is in the visible range is approximately 0.1 to 0.13. However, the thicknesses of films based on DSCG can be varied over a wide range, thus making possible the preparation of films with desired phase-retarding properties despite low anisotropic characteristics of the material. These films are considered in greater detail in T. Fiske, et al., Molecular Alignment in Crystal Polarizers and Retarders, Society for Information Display, Int. Symp. Digest of Technical Papers, Boston, Mass., May 19-24 (2002), pp. 566-569. The main disadvantage of many of these is their dynamic instability, which leads to gradual recrystallization of the LC molecules and degradation of the anisotropy.

Other anisotropic materials have been synthesized based on water-soluble organic dyes utilizing the above-mentioned technology; see, e.g., U.S. Pat. Nos. 5,739,296 and 6,174,394 and European patent EP 0961138. These materials exhibit high optical absorption in the visible spectral range, which limits their application to the manufacture of transparent birefringent films.

Still other anisotropic materials have been synthesized based on acenaphtho[1,2-b]quinoxaline sulfoderivatives having the general structural formula

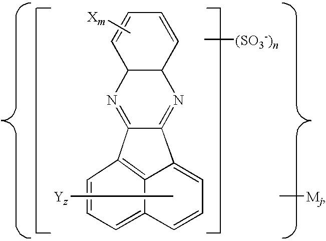

where n is an integer in the range from 1 to 4; m is an integer in the range from 0 to 4; z is an integer in the range from 0 to 6; m+z+n≦10; X and Y are molecular fragments individually selected from the list including CH₃, C₂H₅, OCH₃, OC₂H₅, Cl, Br, OH, OCOCH₃, NH₂, NHCOCH₃, NO₂, F, CF₃, CN, OCN, SCN, COOH, and CONH₂; M is a counter ion; and j is the number of counter ions in the molecule, with a proviso that, when n=1 and SO₃— occupies position 1, then m≠0 or z≠0.

It has been found that an LLC system can be obtained comprising at least one acenaphtho[1,2-b]quinoxaline sulfoderivative having the structure of any one or a combination of

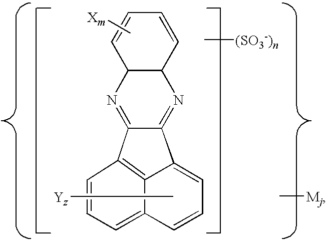

where n is an integer in the range from 1 to 4; m is an integer in the range from 0 to 4; z is an integer in the range from 0 to 6; m+z+n≦10; X and Y are molecular fragments individually selected from the list including CH₃, C₂H₅, OCH₃, OC₂H₅, Cl, Br, OH, OCOCH₃, NH₂, NHCOCH₃, NO₂, F, CF₃, CN, OCN, SCN, COOH, and CONH₂; M is a counter ion; and j is the number of counter ions in the molecule.

The disadvantage of this prior art system is low environmental stability of the crystalline film and high degree of depolarisation of light that propagated through the film with polycrystalline structure. Yet another disadvantage is a tendency of the crystalline film to re-crystallization under high humidity conditions that increases scattering and depolarisation of propagating light.

Thus, there is a general need for films which are optically anisotropic and sufficiently transparent in the spectral regions in which they are Intended to operate. In particular, there is a need for such optical films which are transparent in the visible range. As used herein, the “visible range” has a lower boundary that is approximately equal to 400 nm, and an upper boundary that is approximately equal to 700 nm. The upper boundary of the UV spectral range is lower than the lower boundary of the visible range.

It is therefore desirable to provide improved methods for the synthesis and manufacture of anisotropic films. It is also desirable to provide optical films, which are resistant to humidity and temperature variations.

In the first aspect, the present invention provides an acenaphthoquinoxaline sulfonamide heterocyclic derivative of the general structural formula

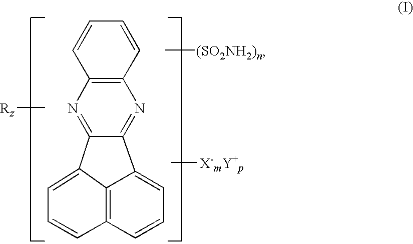

where n is 1, 2 or 3; X is an acid group [Alla—for clarity, the term acid group should be further defined, preferably in the claim but at least in the description. A sub-claim directed to an intermediate generalisation (e.g. X is —COO⁻, —SO3⁻ . . . ) should preferably also be included. AS: Dependent claims 3 and 5 correspond to the specific cases when an acid group is carboxylic or sulfonic. We would like to keep it this way and narrow Claim 1 during the prosecution if required. Theoretically we can add for example HRPO4, H(PO4)2, where R is alkyl or aryl. Otherwise we can add the dependent claim right after the independent claim on the synthesis of the compound that will have “ . . . the acid group consisting of . . . ” and list all four acid groups we can think of. Let us do so if this is advisable.]; m is 1, 2 or 3; Y is a counterion selected from the list consisting of H⁺, NH₄⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, and —CONH₂; and z is 1, 2, 3 or 4.

The acenaphthoquinoxaline sulfonamide heterocyclic derivative is substantially transparent for electromagnetic radiation in the visible spectral range. A solution of this acenaphthoquinoxaline sulfonamide derivative is capable of forming a substantially transparent optical crystal layer on a substrate, with the heterocyclic molecular planes oriented predominantly substantially perpendicularly to the substrate surface.

The present invention provides a practical solution by meeting the needs for a compensator by creating crystalline retarder films with high optical parameters on the basis of new organic compounds.

Sulfonamide groups have capacity to form strong hydrogen bonds (H-bonds). Sulfonamide groups are two times more susceptible to H-bond formation than sulfonate groups. This property of sulfonamide groups strengthens the formation of strong molecular stacks and increases stability of a resulting film. The films formed by organic compounds comprising sulfonamide groups have stable crystalline structure, low sensitivity to humidity variations and higher optical characteristics due to coating uniformity. In addition, such films are not susceptible to recrystaillzation.

In the second aspect, the present invention provides an optical crystal film on a substrate with front and rear surfaces, the film comprising at least one organic layer comprising at least one acenaphthoquinoxaline sulfonamide derivative salt of the general structural formula

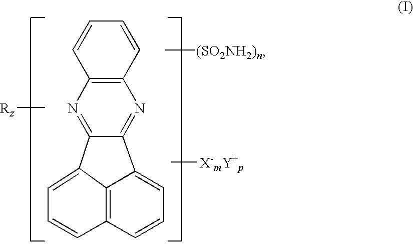

where n is 1, 2 or 3; X is an acid group; m is 1, 2 or 3; Y is a counterion selected from the list consisting of H⁺, NH₄⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, and —CONH₂; and z is 1, 2, 3 or 4.

The conjugated heterocyclic molecular planes of said acenaphthoquinoxaline sulfonamide derivative are oriented predominantly substantially perpendicularly to the substrate surface. Said organic layer is substantially transparent for electromagnetic radiation in the visible spectral range.

In the third aspect, the present Invention provides a method for manufacturing an optical crystal film on a substrate, which comprises the steps of: (1) the application to a substrate of a solution of an acenaphthoquinoxaline sulfonamide derivative, or a combination of such derivatives, of the general structural formula

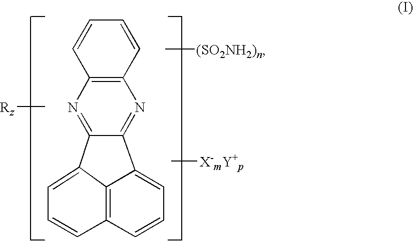

where n is 1, 2 or 3; X is an acid group; m is 1, 2 or 3; Y is a counterion selected from the list consisting of H⁺, NH₄⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, and —CONH₂; and z is 1, 2, 3 or 4, wherein said solution is substantially transparent for electromagnetic radiation in the visible spectral range from approximately 400 to approximately 700 nm; and (2) drying to form a solid crystalline layer.

The general description of the present invention having been made, a further understanding can be obtained by reference to the specific preferred embodiments, which are given herein only for the purpose of illustration and are not intended to limit the scope of the appended claims.

The present invention relates to the synthesis of heterocyclic organic compounds suitable for manufacturing optical films on substrates, in which the molecular planes are oriented predominantly substantially perpendicular to the substrate surface. The heterocyclic compounds comprise at least one group providing water-solubility (said at least one group preferably being a sulfo- or carboxylic group) and at least one group providing H-bonding along the supramolecular stacks (said at least one group preferably being a sulfonamide group).

Thus, the present invention provides an acenaphthoquinoxaline sulfonamide heterocyclic derivative of the general structural formula

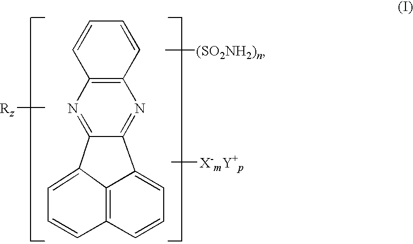

where n is 1, 2 or 3; X is an acid group; m is 1, 2 or 3; Y is a counterion selected from the list consisting of H⁺, NH₄⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, and —CONH₂; and z is 1, 2, 3 or 4. Said acenaphthoquinoxaline sulfonamide derivative is substantially transparent for electromagnetic radiation in the visible spectral range from approximately 400 to approximately 700 nm. By using a solution of the acenaphthoquinoxaline sulfonamide derivative, it is possible to obtain an optical crystal film with the heterocyclic molecular planes oriented predominantly substantially parallel to the substrate surface.

Preferably, X is selected from the group consisting of —COO⁻, —SO₃⁻, and phosphorous-containing acid groups, for example —HPO₄⁻, —RPO₄⁻, —HPO₃⁻ and —RPO₃⁻ wherein R is alkyl or aryl, for example C1-C6 alkyl (branched or unbranched), phenyl or tolyl.

In certain embodiments of the disclosed invention, said acenaphthoquinoxaline sulfonamide derivative absorbs electromagnetic radiation in at least one predetermined subrange of the UV spectral range. The molecules of acenaphthoquinoxaline sulfonamide derivative can absorb electromagnetic radiation only in a part of the UV spectral range, rather than in the entire range, and this part of the UV range will be called subrange. This subrange can be determined experimentally for each particular acenaphthoquinoxaline sulfonamide derivative. In certain embodiments of the disclosed acenaphthoquinoxaline sulfonamide derivative, at least one of said 1, 2 or 3 acid groups is a carboxylic group. Examples of acenaphthoquinoxaline sulfonamide derivatives containing carboxylic groups and having general structural formulas corresponding to structures 1-7 are given in Table 1.

TABLE 1
Examples of acenaphthoquinoxaline sulfonamide
derivatives containing carboxylic groups

| Structure | # |
|---|---|
| 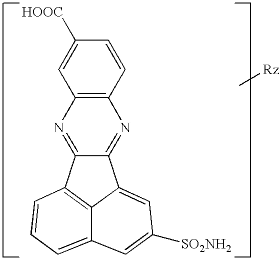 | 1 |
| 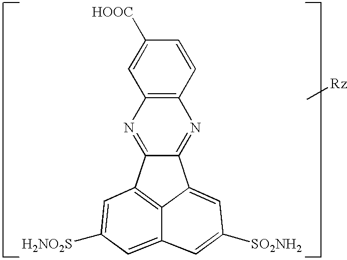 | 2 |
| 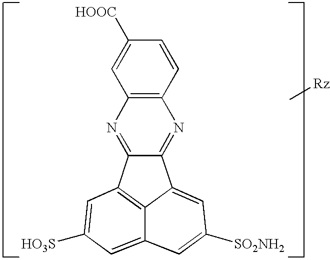 | 3 |
| 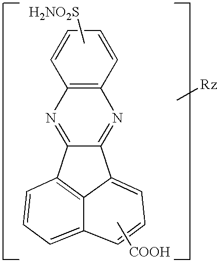 | 4 |
| 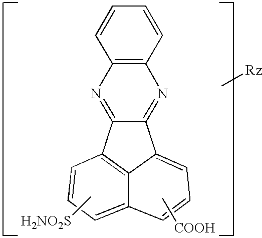 | 5 |
| 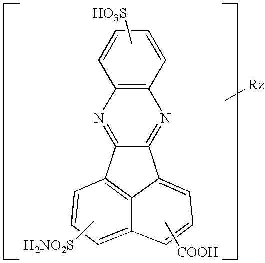 | 6 |
| 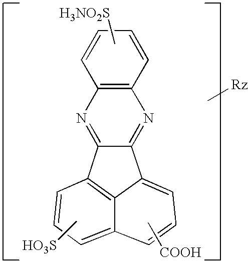 | 7 |

In further embodiments of the disclosed acenaphthoquinoxaline sulfonamide derivative, at least one of said 1, 2 or 3 acid groups is a sulfonic group. Examples of acenaphthoquinoxaline sulfonamide derivatives containing sulfonic groups and having general structural formulas corresponding to structures 8-13 are given in Table 2.

TABLE 2
Examples of acenaphthoquinoxaline sulfonamide
derivatives containing sulfonic groups

| Structure | # |
|---|---|
| 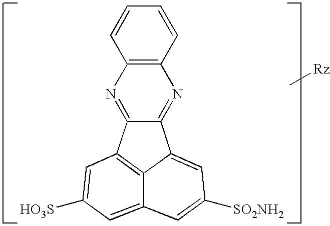 | 8 |
| 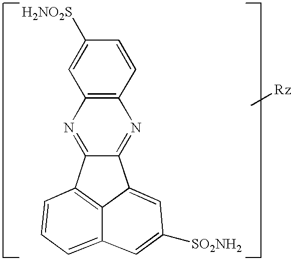 | 9 |
| 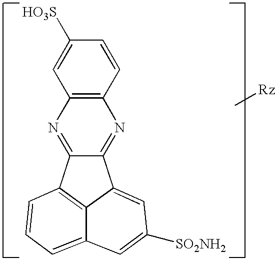 | 10 |
| 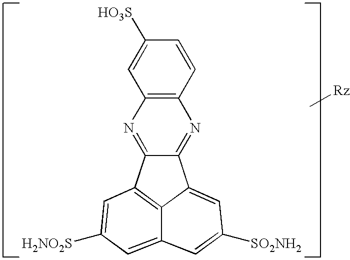 | 11 |
| 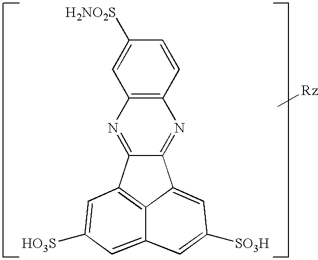 | 12 |
| 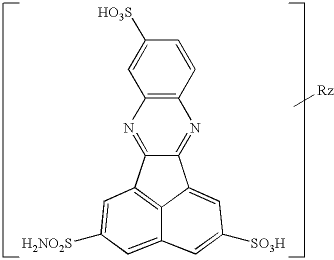 | 13 |

In a second aspect, the present invention provides an optical crystal film on a substrate having front and rear surfaces, the film comprising at least one organic layer containing at least one acenaphthoquinoxaline sulfonamide derivative of the general structural formula

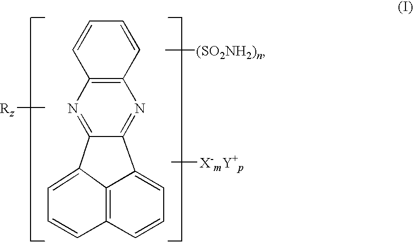

where n is 1, 2 or 3; X is an acid group; m is 1, 2 or 3; Y is a counterion selected from the list consisting of H⁺, NH₄⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, and —CONH₂; and z is 1, 2, 3 or 4. The conjugated heterocyclic molecular planes of said acenaphthoquinoxaline sulfonamide derivatives are oriented predominantly substantially perpendicularly to the substrate surface. Said organic layer is substantially transparent for electromagnetic radiation in the visible spectral range.

Preferably, X is selected from the group consisting of —COO⁻, —SO₃⁻, and phosphorous-containing acid groups, for example —HPO₄⁻, —RPO₄⁻, —HPO₃⁻ and —RPO₃⁻ wherein R is alkyl or aryl, for example C1-C6 alkyl (branched or unbranched), phenyl or tolyl.

In certain embodiments of the disclosed optical crystal film, said organic layer absorbs electromagnetic radiation in at least one predetermined spectral subrange of the UV range.

The disclosed optical crystal film can absorb electromagnetic radiation only in a part of the UV spectral range, rather than In the entire range, and this part of the UV range will be called subrange. This subrange can be determined experimentally for each particular solution of an acenaphthoquinoxaline sulfonamide derivative that is used for the formation of the optical crystal film. Similarly, the absorption subrange can be experimentally determined for a mixture of acenaphthoquinoxaline sulfonamide derivative used for the formation of said film. Thus, such experimentally determined absorption subrange electromagnetic radiation can be considered as the predetermined subrange.

In further embodiments of the disclosed optical crystal film, at least one of the 1, 2 or 3 acid groups is a carboxylic group. Examples of acenaphthoquinoxaline sulfonamide derivatives containing carboxylic groups and having a general structural formula corresponding to structures 1-7 are given in Table 1. In yet further embodiments of the disclosed optical crystal film, at least one of the 1, 2 or 3 acid groups is a sulfonic group. Examples of acenaphthoquinoxaline sulfonamide derivatives containing sulfonamide groups and having a general structural formula corresponding to structures 8-13 are given in Table 2.

The optical crystal film is preferably non-hygroscopic and substantially insoluble in water and/or in water-miscible solvents. A combination of sulphonamide and carboxylic groups in the derivative allows for the production of films that are insoluble in water and non-hygroscopic once they are dry.

A combination of sulphonamide and at least one sulfonic group in the derivative requires treatment with an alkaline earth metal salt solution, for example with an aqueous solution of a Ba⁽²⁺⁾ salt, in order to obtain an insoluble film, but in this case an advantage is also in a low film hygroscopicity and high stability.

The organic layer may contain two or more acenaphthoquinoxaline sulfonamide derivatives with the general structural formula I, each ensuring the absorption of electromagnetic radiation in at least one predetermined wavelength subrange of the UV spectral range. In certain embodiments of the optical crystal film, said acenaphthoquinoxaline sulfonamide derivatives form stacks oriented predominantly substantially parallel to the substrate surface.

Designations of refraction indices convenient for the disclosed invention and connected with optical crystal film will be used below: one refraction index (nz) in the normal direction to the substrate surface and two refraction indices (nx and ny) corresponding to two mutually perpendicular directions in the plane of the substrate surface. The following designations for absorption coefficients will be used also: kx, ky, and kz.

In another embodiment of the optical crystal film according to this invention, said organic layer is a biaxial retardation layer possessing one refraction index (nz) in the normal direction to the substrate surface and two refraction Indices (nx and ny) corresponding to two mutually perpendicular directions in the plane of the substrate surface. In certain embodiments, the refractive indices nx, ny and nz obey the following condition: nx<ny<nz. In further embodiments of the optical crystal film, the in-plane refraction indices (nx and ny) and the organic layer thickness d obey the following condition: d·(ny−nx)<20 nm. In yet further embodiments, the in-plane refractive indices (nx and ny) and the organic layer thickness d obey the following condition: d·(ny−nx)<10 nm. In yet further embodiments, the in-plane refractive indices (nx and ny) and the organic layer thickness d obey the following condition: d·(ny−nx)<5 nm.

In alternative embodiments, the refractive indices nx, ny and nz obey the following condition: nx>nz>ny. In certain embodiments of the optical crystal film, the refractive indices nx and nz and the organic layer thickness d obey the following condition: d·(nx−nz)<20 nm. In yet further embodiments, the refractive indices nx and nz and the organic layer thickness d obey the following condition: d·(nx−nz)<10 nm. In yet further embodiments, the refractive Indices nx and nz and the organic layer thickness d obey the following condition: d·(nx−nz)<5 nm.

The substrate is preferably transparent for electromagnetic radiation in the visible spectral range. The substrate may comprise a polymer, for example PET (polyethylene terephthalate). In alternative embodiments of the disclosed optical crystal film, the substrate comprises a glass. In one embodiment of the disclosed optical crystal film, the transmission coefficient of the substrate does not exceed 2% at any wavelength in the UV spectral range. In another embodiment of the optical crystal film, the transmission coefficient of the substrate in the visible spectral range is not less than 90%.

In still another possible embodiment of the disclosed optical crystal film, the rear surface of the substrate is covered with an additional antireflection or antiglare coating. In another embodiment of the disclosed invention, the rear surface of the substrate further contains a reflective layer.

The disclosed invention also provides an optical crystal film further comprising an additional adhesive transparent layer placed on said reflective layer. In another embodiment of the invention, the optical crystal film further comprises an additional transparent adhesive layer placed on top of the optical crystal film. In one embodiment of the disclosed invention, the optical crystal film further comprises a protective coating formed on the adhesive transparent layer.

In certain embodiments of the optical crystal film, the substrate is a specular or diffusive reflector. In another embodiment of the optical crystal film, the substrate is a reflective polarizer. In still another embodiment, the optical crystal film further comprises a planarization layer deposited onto the front surface of the substrate. In yet another embodiment of the invention, the optical crystal film further comprises an additional transparent adhesive layer placed on top of the organic layer. In another possible embodiment of the invention, the optical crystal film further comprises an additional transparent adhesive layer placed on top of the optical crystal film. In one embodiment of the disclosed invention, the optical crystal film further comprises a protective coating formed on the adhesive transparent layer.

In the embodiments of the disclosed optical crystal film wherein the adhesive layer is present, the transmission coefficient of the adhesive layer does not exceed 2% at any wavelength in the UV spectral range. In another embodiment of the disclosed optical crystal film, the transmission coefficient of the adhesive layer in the visible spectral range is not less than 90%.

In still another embodiment of the disclosed invention the optical crystal film comprises two or more organic layers, wherein each of these layers contains different acenaphthoquinoxaline sulfonamide derivatives of the general structural formula I, each of which absorb electromagnetic radiation in at least one predetermined wavelength subrange of the UV spectral range.

In another aspect, the present invention provides a method for the manufacture of optical crystal films on a substrate, which comprises the steps of (1) applying to a substrate a solution of an acenaphthoquinoxaline sulfonamide derivative, or a combination of such derivatives of the general structural formula

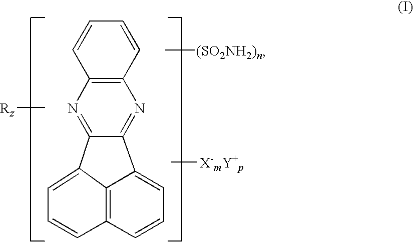

wherein n is 1, 2 or 3; X is an acid group; m is 1, 2 or 3; Y is a counterion selected from the list consisting of H⁺, NH₄⁺, Na⁺, K⁺, and Li⁺; p is the number of counterions providing neutral state of the molecule; R is a substituent selected from the list consisting of —CH₃, —C₂H₅, —NO₂, —Cl, —Br, —F, —CF₃, —CN, —OH, —OCH3, —OC₂H₅, —OCOCH₃, —OCN, —SCN, —NH₂, —NHCOCH₃, and —CONH₂; and z is 1, 2, 3 or 4, and wherein said solution is substantially transparent for electromagnetic radiation in the visible spectral range from approximately 400 to approximately 700 nm;

and (2) drying to form a solid crystalline layer.

In one embodiment of the disclosed method, said method further comprises the step of applying an external alignment action upon the solution prior to the drying step. The external alignment action can be produced by mechanical forces such as a shearing force applied when the solution is spread on the surface by the tool, comprising a knife-like doctor, a Mayer rod (a cylindrical rod wound with a wire), a slot-die or any other technique known in the art. Besides mechanical forces, one can use an application of electrical, electro-magnetical, gravitational forces or any others which allow orienting of the film on the substrate in the mode required. The external alignment can be applied at the same time as the application of the solution to the substrate, or after the application of the solution but before the drying step.

In those embodiments wherein at least one acid group X is SO₃⁻, the method comprises the additional step of treating the film with an alkaline earth metal salt solution, for example with a Ba⁽²⁺⁾ salt.

The present invention provides a simple and inexpensive method for fabricating organic crystal films with phase-retarding properties, in particular optical retarders or compensators such as A-plates. The present invention also provides a method of substrate coating via printing from solutions. The present invention also provides the ability to increase the stability of the films due to stack-strengthening with additional hydrogen bonds without increasing the solubility of molecules and hygroscopicity of the resulting films. Further, a low concentration of a liquid crystal solution used for the LLC phase formation provides for the possibility of manufacture of thin optical films. The present invention also provides a method of formation of water-insoluble thin optical films. The layers produced with carboxysulfonamide derivatives are water-insoluble immediately after drying. The films based on other disclosed materials, for example those having at least one sulfonic group in the compound, will undergo a treatment with alkaline earth metal salt solutions. The present invention also provides a low sensitivity of the film material to humidity, which ensures high environmental stability of the obtained films.

In another embodiment of the disclosed method, said solution also ensures the absorption maximums of electromagnetic radiation in at least one predetermined wavelength subrange of the UV spectral range. The solution can absorb electromagnetic radiation only in a part of the UV spectral range, rather than In the entire range, and this part of the UV range will be called subrange. This subrange can be determined experimentally for each particular solution of an acenaphthoquinoxaline sulfonamide derivative that is used for the formation of the optical crystal film. Similarly, the absorption subrange can be experimentally determined for a mixture of acenaphthoquinoxaline sulfonamide derivative used for the formation of said film. Thus, such experimentally determined absorption subrange electromagnetic radiation can be considered as the predetermined subrange.

In certain embodiments, at least one of the 1, 2 or 3 acid groups is a carboxylic group. Examples of acenaphthoquinoxaline sulfonamide derivatives containing carboxylic groups and having a general structural formula corresponding to structures 1-7 are given in Table 1. In other embodiments of the disclosed optical crystal film, at least one of the 1, 2 or 3 acid groups is a sulfonic group. Examples of acenaphthoquinoxaline sulfonamide derivatives containing sulfonamide groups and having a general structural formula corresponding to structures 8-13 are given in Table 2.

In one embodiment of the disclosed method, said solution is based on water (i.e. an aqueous solution) and/or water-miscible solvents. In still another embodiment of the disclosed method, the applied solution layer is dried in airflow and/or elevated temperature preferably in the range of 23-60° C. This temperature range prevents a recrystallization and a shattering (or spotting) of the solid layer. In a possible embodiment of the disclosed method, the substrate is pretreated so as to provide surface hydrophilization before application of said solution. In another embodiment of the present invention, the Ba²⁺ salt is any water-soluble inorganic salt with a Ba⁺⁺ cation. In one possible embodiment of the disclosed method, said solution is a lyotropic liquid crystal solution. In one possible embodiment of the disclosed method, the application of said acenaphthoquinoxaline sulfonamide derivative solution onto the substrate is accompanied or followed by an external orienting action upon this solution. In yet another embodiment of the disclosed method, the method steps are repeated at least once, such that a plurality of solid layers are formed using either the same or different solutions, which absorb electromagnetic radiation in at least one predefined spectral subrange of the UV spectral range.

Other objects and advantages of the present invention will become apparent upon reading detailed description of the examples and the appended claims provided below, and upon reference to the drawings, in which:

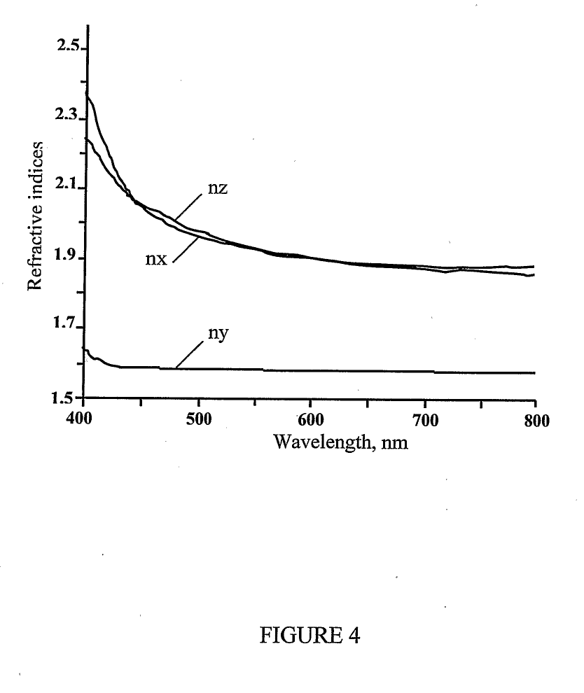

FIG. 4 shows the refractive indices of the organic layer prepared from a mixture of 9-carboxy-acenaphthoquinoxaline-2-sulfonamide and 9-carboxy-acenaphthoquinoxaline-5-sulfonamide (6.0% solution) on a glass substrate.

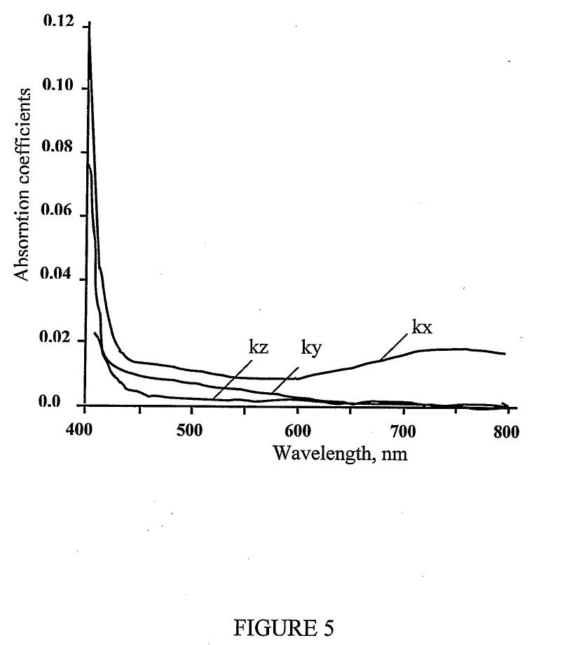

FIG. 5 shows the absorption coefficients of the organic layer prepared from a mixture of 9-carboxy-acenaphthoquinoxaline-2-sulfonamide and 9-carboxy-acenaphthoquinoxaline-5-sulfonamide (6.0% solution) on a glass substrate.

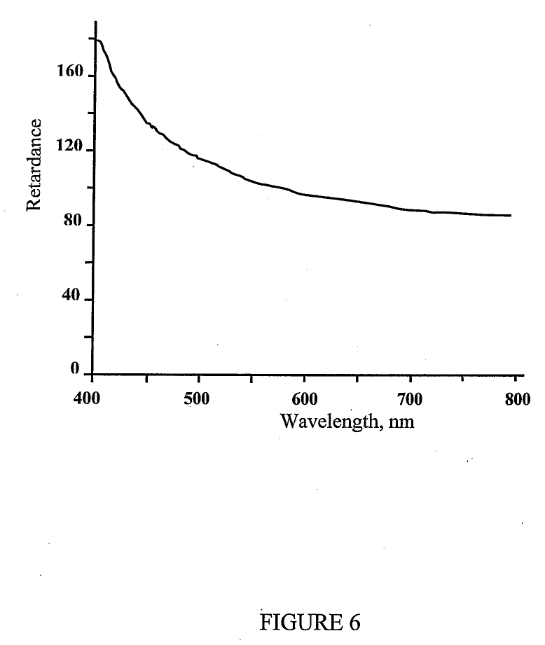

FIG. 6 shows the retardance of the organic layer with a thickness of 312.1 nm prepared from a mixture of 9-carboxy-acenaphthoquinoxaline-2-sulfonamide and 9-carboxy-acenaphthoquinoxaline-5-sulfonamide (6.0% solution) on a glass substrate.

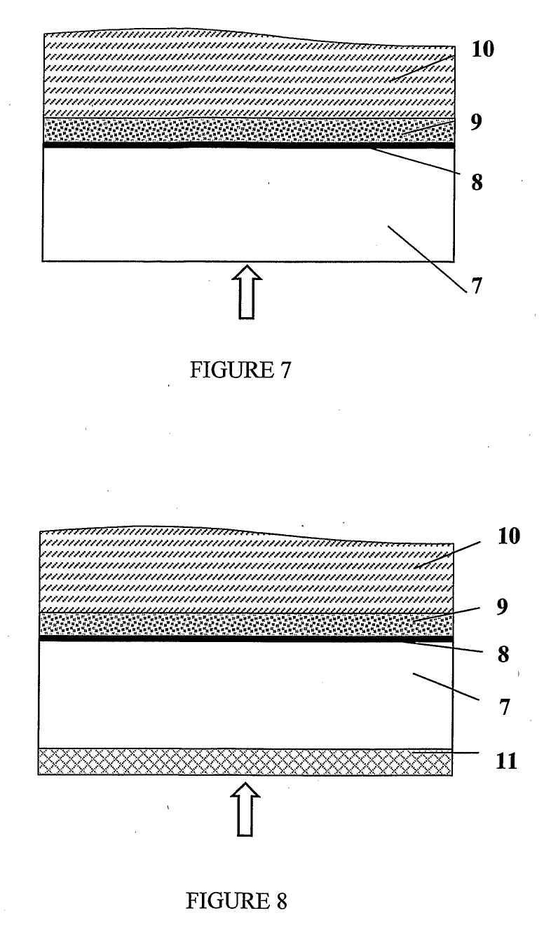

FIG. 7 shows the cross section of an optical crystal film on a substrate, together with additional adhesive and protective layers.

FIG. 8 shows the cross section of an optical crystal film with an additional antireflection layer.

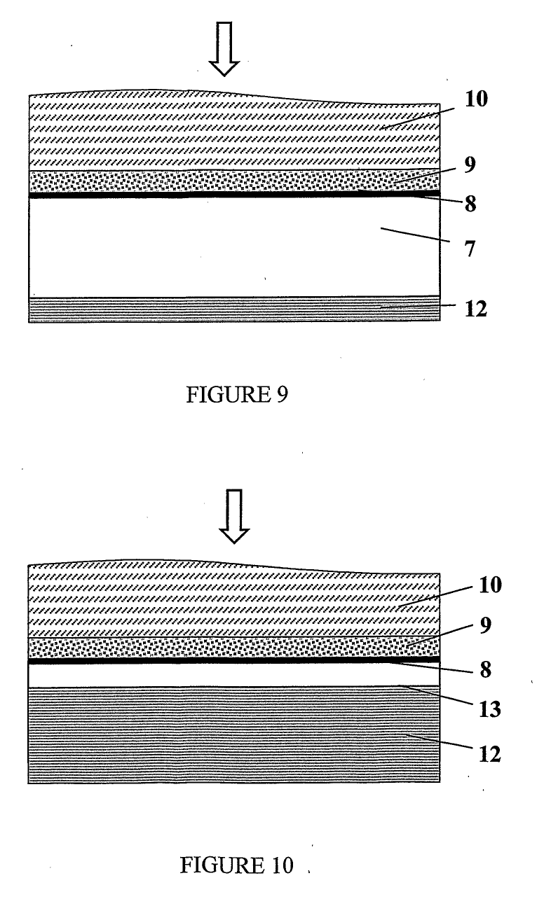

FIG. 9 shows the cross section of an optical crystal film with an additional reflective layer.

FIG. 10 shows the cross section of an optical crystal film with a diffuse or specular reflector as the substrate.

In order that the invention may be more readily understood, reference is made to the following examples, which are intended to be illustrative of the invention, but are not intended to be limiting in scope.

### EXAMPLE 1

The first example describes syntheses of a mixture of 9-carboxy-acenaphthoquinoxaline-2-sulfonamide and 9-carboxy-acenaphthoquinoxaline-5-sulfonamide

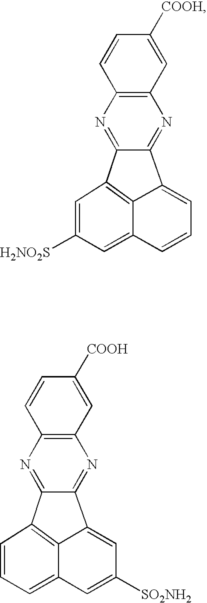

A. Synthesis of 9-carboxy-acenaphthoquinoxaline

A solution of 3,4-diaminobenzoic acid hydrochloride (1.88 g, 0.01 mol) in 75 ml of water was added to the suspension of acenaphthoquinone (1.82 g, 0.01 mol) in 80 ml of acetic acid. The reaction mixture was heated to 95-100° C., treated at this temperature for 15 min with stirring, and cooled. The precipitate was separated by filtration and washed with acetic acid. The final product yield was 2.6 g (87%). Mass spectrum (VISION 2000 spectrometer, negative ion reflection mode): m/z, 298.5; mol. wt, 298.29; electronic absorption spectrum (Ocean PC 2000 spectrometer, aqueous solution of ammonium salt): λₘₐₓ₁=235 nm, and λₘₐₓ₂=320 nm.

B. Synthesis of the Mixture of 9-carboxy-acenaphthoquinoxaline-2-sulfonic acid and 9-carboxy-acenaphthoquinoxaline-5-sulfonic acid

9-Carboxy-acenaphthoquinoxaline (2.0 g, 0.0067 mol) was added to 20 ml of 30% oleum and kept with stirring for 3.5 h at 80-90° C. Then, the reaction mixture was diluted with 36 ml of water and the precipitate was separated by filtration, reprecipitated from acetic acid (100 ml), filtered, and washed with acetone. The final product yield was 1.92 g (76%). Mass spectrum (VISION 2000 spectrometer, negative ion reflection mode): m/z, 377.1; mol. wt. 378.36; electronic absorption spectrum (Ocean PC 2000 spectrometer, aqueous solution of ammonium salt): λₘₐₓ₁=235 nm, and λₘₐₓ₂=320 nm.

C. Synthesis of the Mixture of Chlorides of 9-carboxy-acenaphthoquinaxaline-2-sulfonic acid and 9-carboxy-acenaphthoquinoxaline-5-sulfonic acid

A mixture of 9-carboxy-acenaphthoquinoxaline-2-sulfonic acid and 9-carboxy-acenaphthoquinoxaline-5-sulfonic acid (1.8 g, 0.0047 mol) was added to chlorosulfonic acid 18 ml). Then, 0.3 g of NaCl was added and the reaction mixture was kept with stirring for 3 hours at 80-85° C., cooled, and poured into 350 g of ice. The precipitate was separated by filtration and washed until neutral pH with ice-cold water. The final product yield was 8-9 g of a filter-cake.

D. Synthesis of the Mixture of 9-carboxy-acenaphthoquinaxaline-2-sulfonamide and 9-carboxy-acenaphthoquinoxaline-5-sulfonamide

The filter-cake of the mixture of chlorides of 9-carboxy-acenaphthoquinoxaline-2-sulfonic acid and 9-carboxy-acenaphthoquinoxaline-sulfonic acid (8-10 g) was added to 20 ml of ammonia and the mixture was kept at 3-5° C. for 0.5 hour and then stirred under ambient conditions for 0.5 hour. The obtained ammonia solution was filtered and diluted with isopropanol (˜30 ml). The precipitate was separated by filtration and washed on the filter with isopropanol. The final product yield was 1.2 g (67%). Mass spectrum (VISION 2000 spectrometer): m/z, 377.2; mol. wt, 377.37; electron absorption spectrum (Ocean PC 2000 spectrometer, aqueous solution of ammonium salt): λₘₐₓ₁=235 nm, and λₘₐₓ₂=320 nm. Elemental analysis: C, 60.22; H, 2.91; N, 11.11; anal calcd. for C₁₈H₁₀N₂O₃S: C, 60.47; H, 2.94; N, 11.13; O, 16.96; S, 8.50.

### EXAMPLE 2

This example describes the preparation of an organic layer from a lyotropic liquid crystal solution. A mixture of 9-carboxy-acenaphthoquinoxaline-2-sulfonamide and 9-carboxy-acenaphthoquinoxaline-5-sulfonamide (1 g) obtained as described in Example 1 was stirred for 1 h at a temperature of 20° C. in a mixture of 15.0 ml of deionized water with 0.6 ml of a 10% aqueous ammonia solution until a lyotropic liquid crystal solution was formed.

Fisherbrand microscope glass slides were prepared for coating by treating in a 10% NaOH solution for 30 min, rinsing with deionized water, and drying in airflow with the aid of a compressor. The obtained solution was applied at a temperature of 20° C. and a relative humidity of 65% onto the glass plate surface with a Mayer rod #2.5 moved at a linear velocity of 15 mm/s. The film was dried at the same humidity and temperature.

In order to determine the optical characteristics of the organic layer, the optical transmission spectrum was measured in a wavelength range from approximately 400 to approximately 700 nm using a Cary 500 spectrophotometer. The optical transmission of the organic layer was measured using light beams linearly polarized parallel and perpendicular to the coating direction (Tₚₐᵣ and Tₚₑₙ and respectively). The obtained data were used to calculate the refractive indices (nx, ny, and nz) presented in FIG. 4. The obtained organic layer was anisotropic in the plane (nx=1.93, ny=1.58, nz=1.93). The measurements showed extremely small values of the absorption coefficients of the organic layer (kx, ky, and kz, see FIG. 5). The obtained organic layer exhibited retardation shown in the FIG. 6.

### EXAMPLE 3

FIG. 7 shows the cross section of an optical crystal film formed on substrate 7. The film contains organic layer 8, adhesive layer 9, and protective layer 10. The organic layer can be manufactured using the methods described in Example 2. The polymer layer 10 protects the optical crystal film from damage in the course of its transportation.

This optical crystal film is a semiproduct, which can be used as an external retarder in, for example, LCDs. Upon removal of the protective layer 10, the remaining film is applied onto an LCD glass with adhesive layer 9.

The above described optical crystal film with an additional antireflection layer 11 formed on the substrate can be applied to the LCD front surface (FIG. 8). For example, an antireflection layer of silicon dioxide SiO₂ reduces by 30% the fraction of light reflected from the LCD front surface.

### EXAMPLE 6

With the above described optical crystal film applied to the front surface of an electrooptical device or an LCD, an additional reflective layer 12 can be formed on the substrate (FIG. 9). The reflective layer may be obtained, for example, by depositing an aluminium film.

### EXAMPLE 6

In this example, the organic layer 8 is applied onto the diffuse or specular semitransparent reflector 12 that serves as a substrate (FIG. 10). The reflector layer 12 may be covered with the planarization layer 13 (optional). Polyurethane or an acrylic polymer or any other material can be used for making this planarization layer.

1. An acenaphthoquinoxaline sulfonamide heterocyclic derivative of a general structural formula 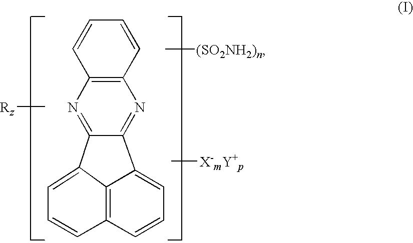
where n is 1, 2 or 3;
X is an acid group;
m is 1, 2 or 3;
Y is a counterion selected from the list consisting of H⁺, NH₄⁺, Na⁺, K⁺, and Li⁺;
p is the number of counterions providing neutral state of the molecule;
R is a substituent selected from the list consisting of —CH₃, —C₂H₅, —NO₂, —Cl, —Br, —F,
—CF₃, —CN, —OH, —OCH₃, —OC₂H₅, —OCOCH₃, —OCN, —SCN, —NH₂, —NHCOCH₃, —CONH₂; and
z is 1, 2, 3 or 4,
wherein said acenaphthoquinoxaline sulfonamide derivative is transparent for incident electromagnetic radiation in the visible spectral range, and a solution of said acenaphthoquinoxaline sulfonamide derivative is capable of forming a substantially transparent optical crystal layer on a substrate, with the heterocyclic molecular planes oriented predominantly substantially perpendicularly to the substrate surface.

2. An acenaphthoquinoxaline sulfonamide derivative according to claim 1 wherein X is selected from the group consisting of —COO⁻, —SO₃⁻, and phosphorous-containing acid groups.

3. An acenaphthoquinoxaline sulfonamide derivative according to claim 2 wherein X is —HPO₄⁻, —RPO₄⁻, —HPO₃⁻ and —RPO₃⁻ wherein R is alkyl or aryl.

4. An acenaphthoquinoxaline sulfonamide derivative according to claim 3 wherein R is C1-C6 branched or unbranched alkyl, phenyl or tolyl.

5. An acenaphthoquinoxaline sulfonamide derivative according to claim 1, which absorbs electromagnetic radiation in at least one predetermined wavelength subrange of the UV spectral range.

6. An acenaphthoquinoxaline sulfonamide derivative according to claim 1, wherein at least one of said 1, 2 or 3 acid groups are selected from the list comprising carboxylic group and sulfonic groups.

7. An acenaphthoquinoxaline sulfonamide derivative according to claim 6 having a general structural formula corresponding to one of structures 1-13:

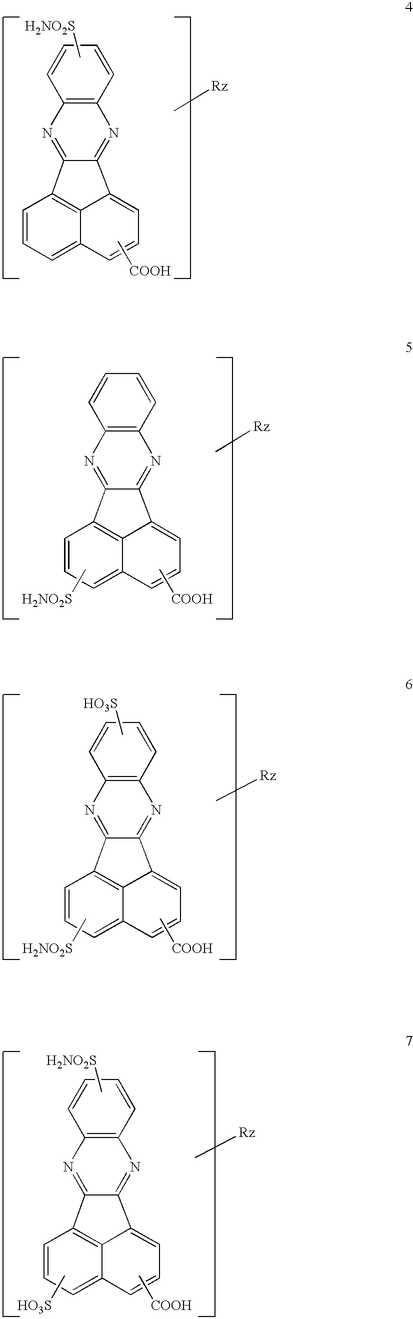

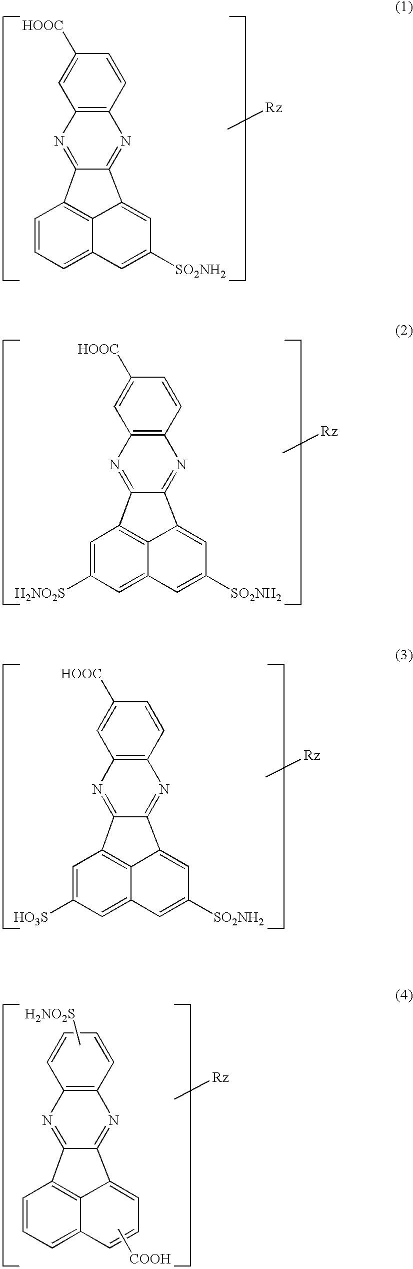

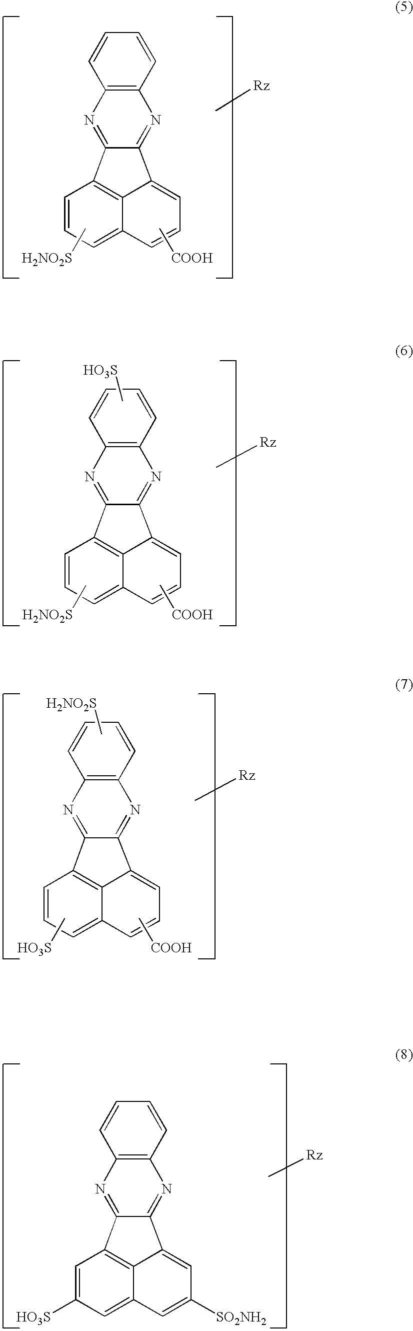

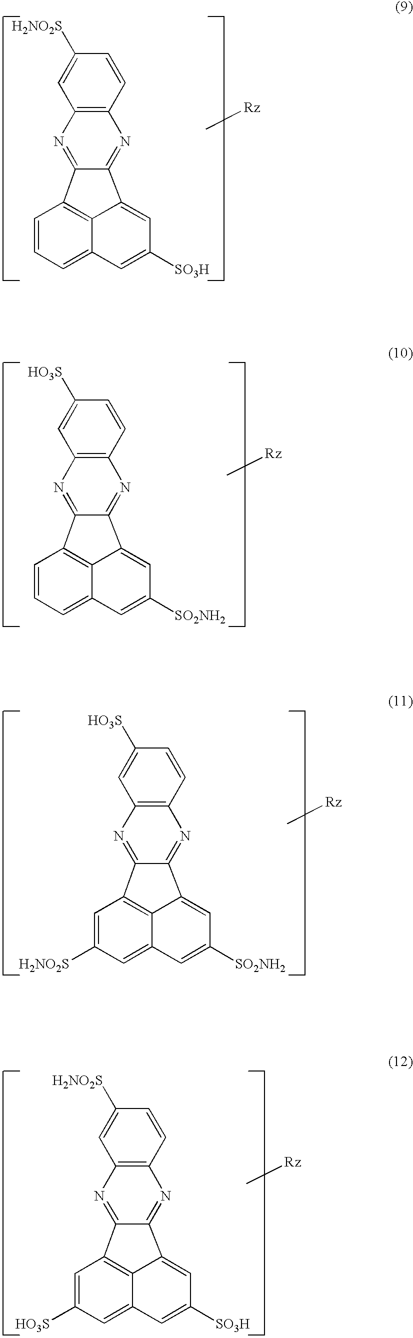

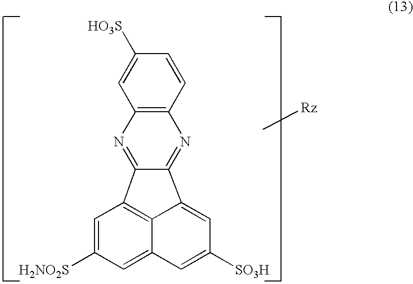

8. An acenaphthoquinoxaline sulfonamide derivative according to claim 6 selected from the group consisting of 9-carboxy-acenaphthoquinoxaline-2-sulfonamide, 9-carboxy-acenaphthoquinoxaline-5-sulfonamide, and a mixture thereof.

9. (canceled)

10. (canceled)

11. (canceled)

12. An optical crystal film on a substrate having front and rear surfaces, the film comprising at least one organic layer applied onto the front surface of the substrate, the organic layer comprising at least one acenaphthoquinoxaline sulfonamide derivative of the general structural formula 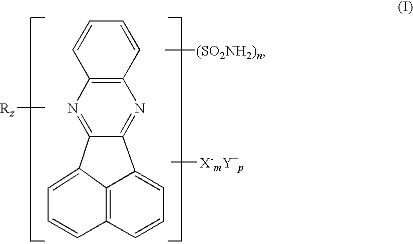
where n is 1, 2 or 3;
X is an acid group;
m is 1, 2 or 3;
Y is a counterion selected from the list consisting of H⁺, NH₄^{+, K}⁺, Li⁺, Ba⁺⁺;
p is the number of counterions providing neutral state of the molecule;
R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, —CONH₂; and
z is 1, 2, 3 or 4,
wherein the planes of the acenaphthoquinoxaline sulfonamide derivative are oriented predominantly substantially perpendicularly to the substrate surface, and
said organic layer is substantially transparent for electromagnetic radiation in the visible spectral range.

13. An optical crystal film according to claim 12 wherein X is selected from the group consisting of —COO⁻, —SO₃⁻, and phosphorous-containing acid groups.

14. An optical crystal film according to claim 13 wherein X is —HPO₄⁻, —RPO₄⁻, —HPO₃⁻ and —RPO₃⁻ wherein R is alkyl or aryl.

15. An optical crystal film according to claim 14 wherein R is C1-C6 branched or unbranched alkyl, phenyl or tolyl.

16. An optical crystal film according to claim 15, wherein said organic layer absorbs electromagnetic radiation in at least one predetermined wavelength subrange of the UV spectral range.

17. An optical crystal film according to claim 12, wherein at least one of said 1, 2 or 3 acid groups of the at least one acenaphthoquinoxaline sulfonamide derivative is selected from the list comprising carboxylic and sulfonic groups.

18. An optical crystal film according to claim 17, wherein said at least one acenaphthoquinoxaline sulfonamide derivative has a general structural formula corresponding to one of structures 1-13:

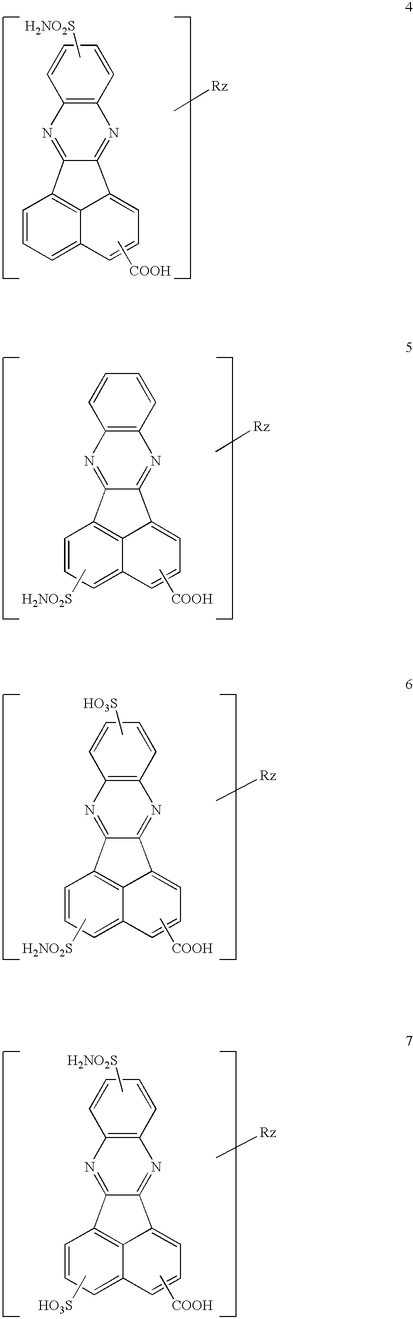

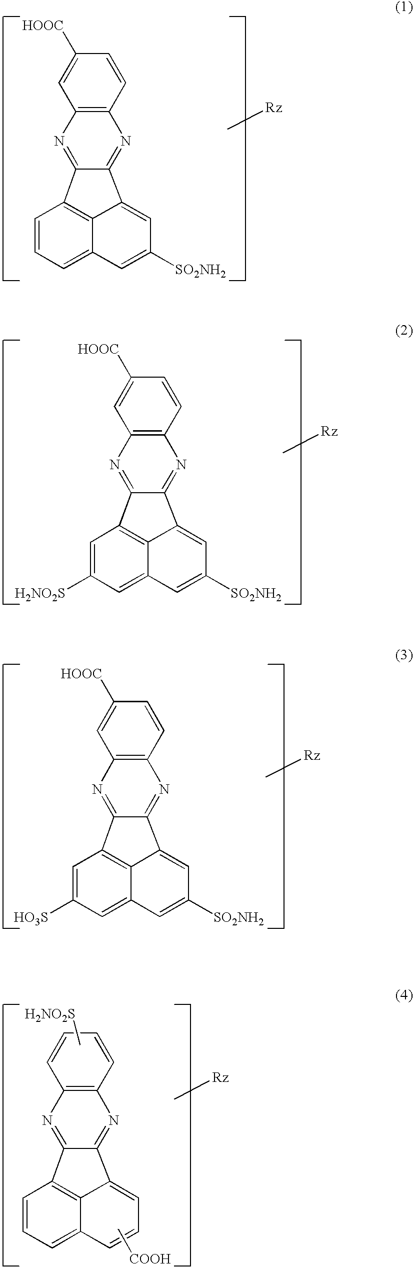

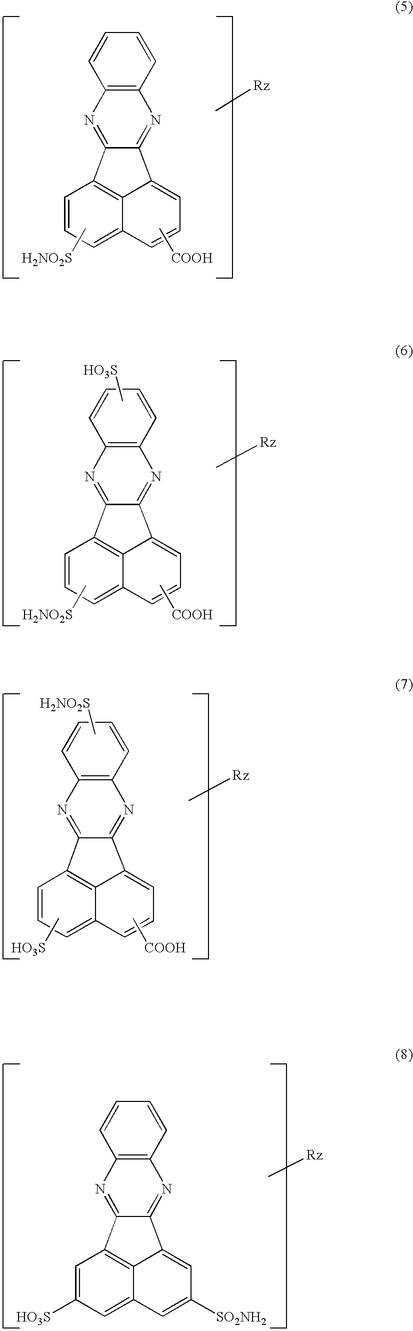

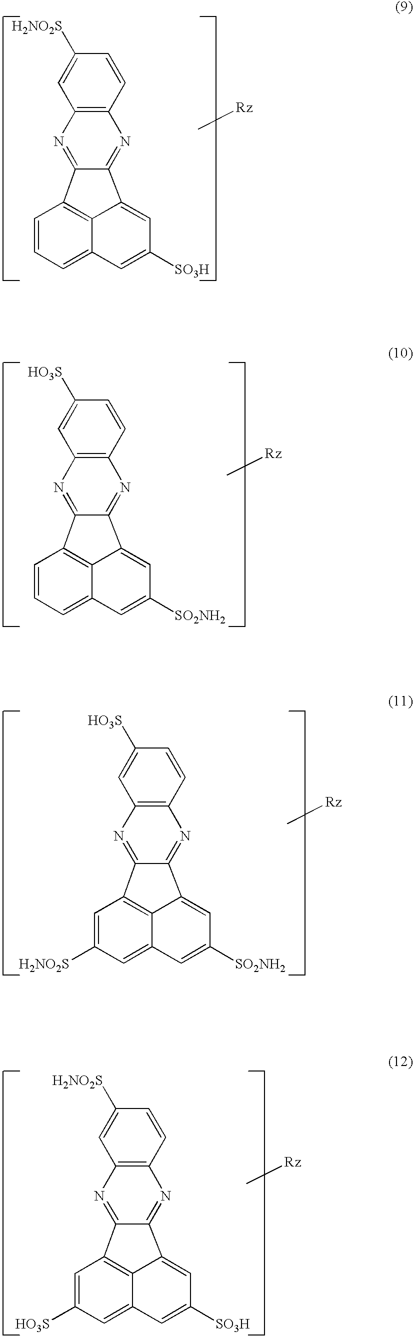

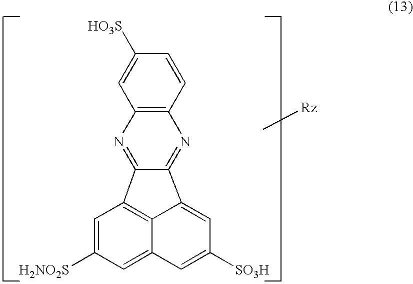

19. An optical crystal film according to claim 17 wherein said at least one acenaphthoquinoxaline sulfonamide derivative is selected from the group consisting of 9-carboxy-acenaphthoquinoxaline-2-sulfonamide, 9-carboxy-acenaphthoquinoxaline-5-sulfonamide, and a mixture thereof.

20. An optical crystal film according to claim 19 wherein said at least one acenaphthoquinoxaline sulfonamide derivative comprises a mixture of 9-carboxy-acenaphthoquinoxaline-2-sulfonamide and 9-carboxy-acenaphthoquinoxaline-5-sulfonamide.

21. (canceled)

22. (canceled)

23. An optical crystal film according to any of claim 12, wherein said crystal film is substantially insoluble in water and/or in water-miscible solvents at a temperature in the range between approximately 18 and 90° C.

24. An optical crystal film according to claim 12, wherein said organic layer comprises two or more acenaphthoquinoxaline sulfonamide derivatives of the general structural formula I, each of which absorb electromagnetic radiation in at least one predetermined wavelength subrange of the UV spectral range.

25. An optical crystal film according to claim 12, wherein said planar molecules of acenaphthoquinoxaline sulfonamide derivatives form stacks oriented predominantly substantially parallel to the substrate surface.

26. An optical crystal film according to claim 12, wherein said organic layer is a biaxial retardation layer possessing one refraction index (nz) in the normal direction to the substrate surface and two refraction indices (nx and ny) corresponding to two mutually perpendicular directions in the plane of the substrate surface.

27. An optical crystal film according to claim 26, wherein the refractive indices nx, ny and nz obey the following condition: nx<ny<nz.

28. An optical crystal film according to claim 27, wherein the in-plane refraction indices (nx and ny) and the organic layer thickness d obey the following condition: d·(ny−nx)<20 nm.

29. (canceled)

30. An optical crystal film according to claim 26, wherein the in-plane refractive indices (nx and ny) and the organic layer thickness d obey the following condition: d·(ny−nx)<5 nm.

31. An optical crystal film according to claim 26, wherein the refractive indices nx, ny and nz obey the following condition: nx>nz>ny.

32. An optical crystal film according to claim 31, wherein the refractive indices nx and nz and the organic layer thickness d obey the following condition: d·(nx−nz)<20 nm.

33. (canceled)

34. An optical crystal film according to claim 26, wherein the refractive indices nx and nz and the organic layer thickness d obey the following condition: d·(nx−nz)<5 nm.

35. An optical crystal film according to claim 12, wherein the substrate is transparent for electromagnetic radiation in the visible spectral range.

36. (canceled)

37. An optical crystal film according to claim 35, wherein the substrate comprises a glass.

38. An optical crystal film according to claim 35, wherein the transmission coefficient of the substrate does not exceed 2% at any wavelength in the UV spectral range.

39. (canceled)

40. An optical crystal film according to claim 28, wherein the rear surface of the substrate has an antireflection or antiglare coating.

41. An optical crystal film according to, claim 28 wherein the rear surface of the substrate has a reflective layer.

42. An optical crystal film according to, claim 12 wherein the substrate is a specular or diffusive reflector.

43. An optical crystal film according to claim 12 wherein the substrate is a reflective polarizer.

44. An optical crystal film according to claim 42, further comprising a planarization layer on the front surface of the substrate.

45. An optical crystal film according to, claim 12, further comprising a transparent adhesive layer on top of the organic layer.

46. An optical crystal film according to claim 45, further comprising a protective coating on the transparent adhesive layer.

47. An optical crystal film according to claim 45, wherein the transmission coefficient of the adhesive layer does not exceed 2% at any wavelength in the UV spectral range.

48. (canceled)

49. An optical crystal film according to claim 12 comprising two or more organic layers, wherein said layers contain different acenaphthoquinoxaline sulfonamide derivatives of the general structural formula I, each of which absorb electromagnetic radiation in at least one predetermined wavelength subrange of the UV spectral range.

50. A method of producing an optical crystal film on a substrate, which comprises the steps of (1) applying a solution of an acenaphthoquinoxaline sulfonamide derivative, or a combination of such derivatives, of the general structural formula 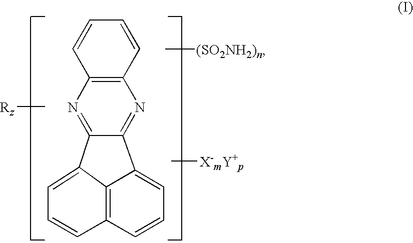
where n is 1, 2 or 3;
X is an acid group;
m is 1, 2 or 3;
Y is a counterion selected from the list consisting of H⁺, NH₄⁺, Na⁺, K⁺, and Li⁺;
p is the number of counterions providing neutral state of the molecule;
R is a substituent selected from the list consisting of —CH₃, —C₂H₅, —NO₂, —Cl, —Br, —F, —CF₃, —CN, —OH, —OCH₃, —OC₂H₅, —OCOCH₃, —OCN, —SCN, —NH₂, —NHCOCH₃, —CONH₂; and
z is 1, 2, 3 or 4,
wherein said solution is substantially transparent for electromagnetic radiation in the visible spectral range, and
(2) drying to form a solid crystalline layer.

51. A method according to claim 50, further comprising the step of applying an external alignment action upon the solution prior to the drying step.

52. (canceled)

53. A method according to claim 50, wherein at least one of said 1, 2 or 3 acid groups of the acenaphthoquinoxaline sulfonamide derivative is selected from the list comprising carboxylic and sulfonic groups.

54. A method according to claim 53, wherein said acenaphthoquinoxaline sulfonamide derivative has a general structural formula corresponding to one of structures 1-13:

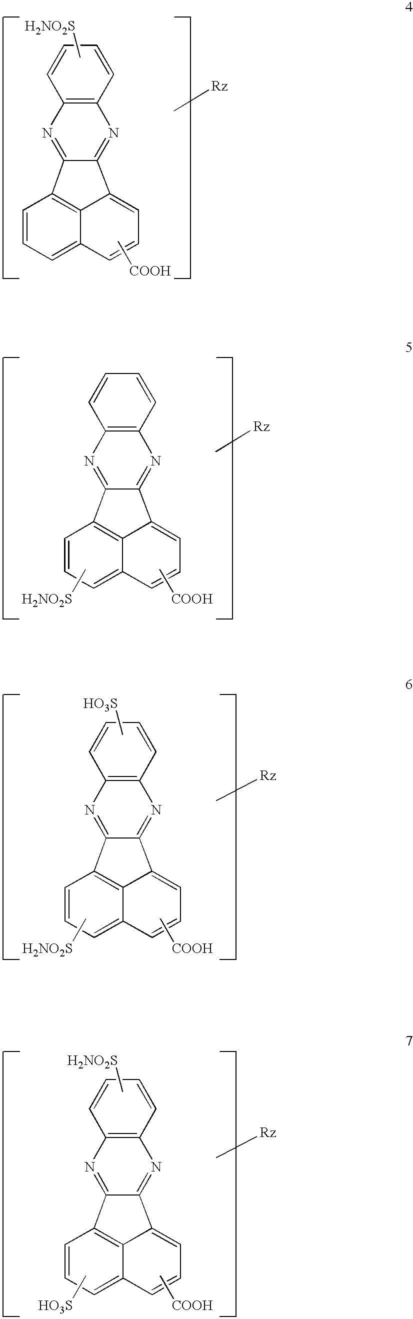

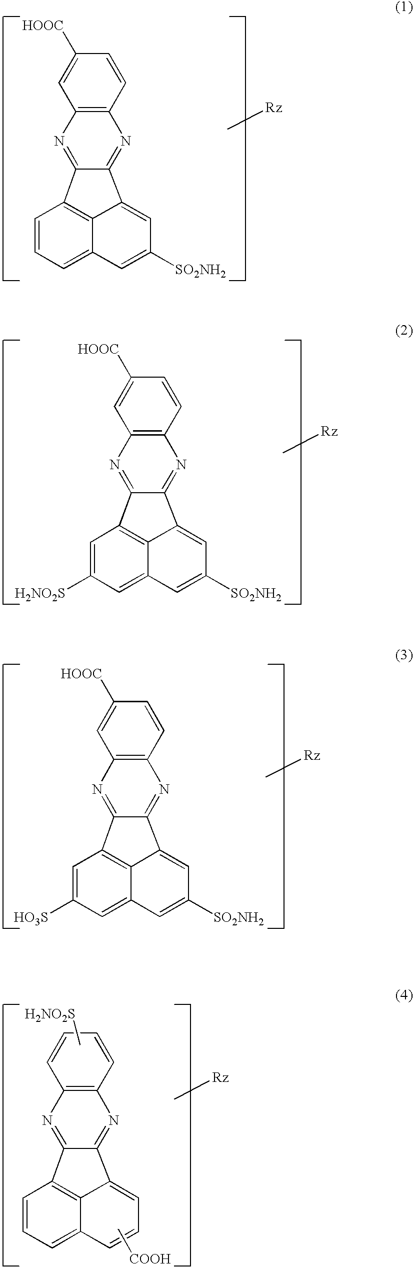

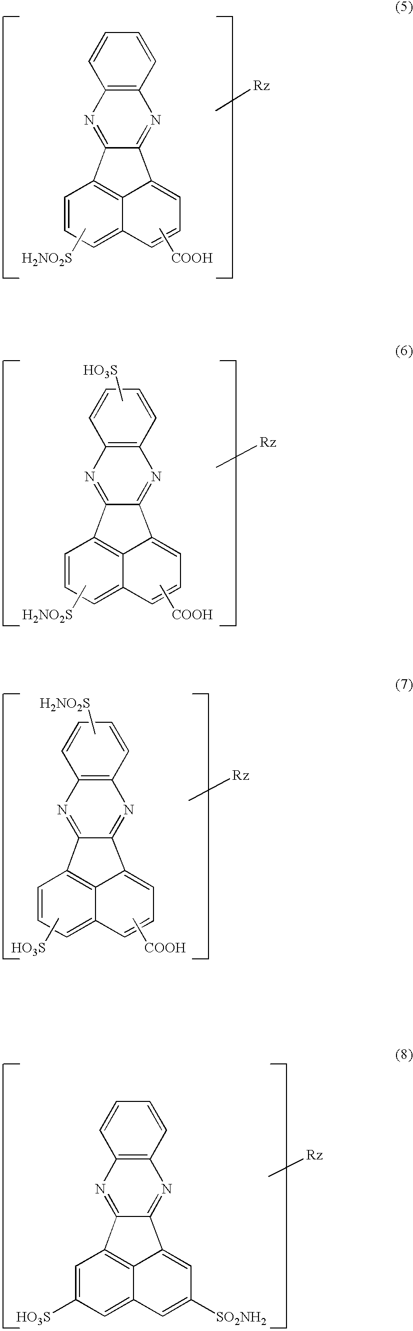

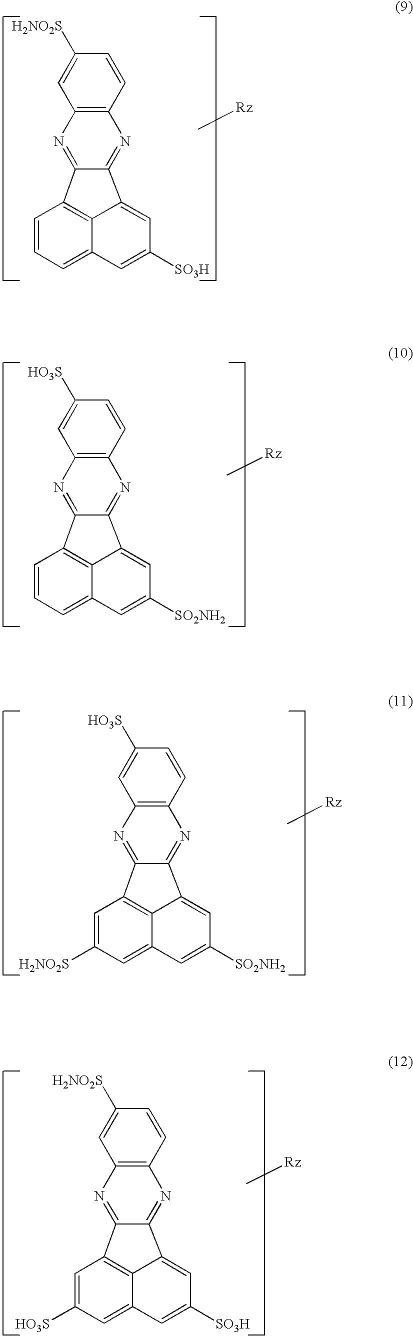

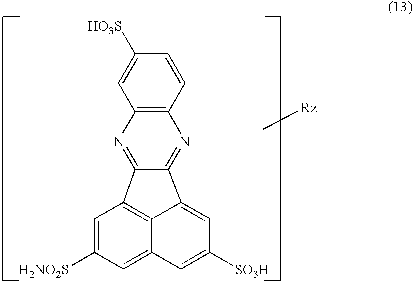

55. A method according to claim 53, wherein the solution of an acenaphthoquinoxaline sulfonamide derivative comprises an acenaphthoquinoxaline sulfonamide derivative selected from the group consisting of 9-carboxy-acenaphthoquinoxaline-2-sulfonamide, 9-carboxy-acenaphthoquinoxaline-5-sulfonamide, and a mixture thereof.

56. A method according to claim 55, wherein the solution of an acenaphthoquinoxaline sulfonamide derivative comprises a mixture of 9-carboxy-acenaphthoquinoxaline-2-sulfonamide and 9-carboxy-acenaphthoquinoxaline-5-sulfonamide.

57. (canceled)

58. (canceled)

59. A method according to claim 50, wherein said solution is based on water and/or water-miscible solvents.

60. A method according to claim 50, wherein the drying step is executed in airflow and/or at elevated temperature.

61. (canceled)

62. A method according to claim 50, wherein the substrate is pre-treated prior to the application of the solution so as to render its surface hydrophilic.

63. A method according to claim 50, further comprising the step of treating the solid layer with a solution of a water soluble inorganic salt with a Ba⁺⁺ cation.

64. A method according to claim 50, wherein said solution is a lyotropic liquid crystal solution.

65. A method according to claim 50, wherein the method steps are repeated at least once, such that a plurality of solid layers are formed using either the same or different solutions.

66. (canceled)

67. (canceled)

68. (canceled)

69. (canceled)
